(19) United States
(12) Patent Application Publication
(10) Pub. No.: US 2009/0163737 A1
(43) Pub. Date: Jun. 25, 2009

(54) NOVEL PROCESS FOR THE PREPARATION OF CHIRAL COMPOUNDS DERIVED FROM HEXANOIC ACID ESTERS AND INTERMEDIATES USED IN THE SYNTHESIS OF CHIRAL 2-(BROMOMETHYL)-2-ETHYLHEXANOIC ACID

(75) Inventors: Veronique CROCQ-STUERGA, Dijon (FR); Patrick ROUSSEL, Thiais (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMA S.A., Antony (FR)

(21) Appl. No.: 12/394,262

(22) Filed: Feb. 27, 2009

Related U.S. Application Data
(63) Continuation of application No. 11/970,113, filed on Jan. 7, 2008.
Application No. 11/970,113 is a continuation-in-part of application No. 10/737,955, filed on Dec. 17, 2003, now abandoned.
(60) Provisional application No. 60/478,048, filed on Jun. 12, 2003.

(30) Foreign Application Priority Data
Dec. 20, 2002 (FR) 0216229

(51) Int. Cl. C07C 69/67; C12P 7/62; C12P 7/40; C07C 59/21

(52) U.S. Cl. 560/174; 435/135; 435/136; 562/577

(57) ABSTRACT

The present invention comprises a novel process for the preparation of a chiral compound of formula (I)

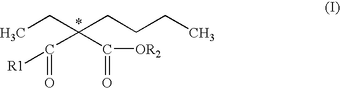

wherein R₁ is hydroxyl or a group which activates the carboxyl and R₂ is alkyl optionally substituted by halogen or benzyl, its preparation, its application in the synthesis of chiral 2-bromomethyl-2-ethylhexanoic acid and novel intermediates.

### FIELD OF THE INVENTION

The present invention relates generally to processes for the preparation of pharmaceutical compounds and compositions for the treatment of metabolic disorders and the disease states that are the direct manifestation thereof such as diabetes, hyperglycemia, hypoglycemia and the like. More specifically, the present invention comprises novel processes for the preparation of novel chiral intermediates useful in the subsequent preparation of therapeutically effective bile acid reabsorption inhibitors.

### BACKGROUND OF THE INVENTION

Recent advances have provided important new information on the physiological mechanisms of bile acid transport and metabolism. Bile acids, which are essential for the digestion and absorption of lipids and lipid-soluble vitamins, are metabolic products of cholesterol and are a major regulator of cholesterol homeostasis. Bile acids are pharmacologically interesting as potential carriers of liver-specific drugs, absorption enhancers and as new cholesterol-lowering agents. Furthermore, the tools of molecular recognition and combinatorial chemistry have been used to explore the drug discovery possibilities of bile acids.

Serum cholesterol is regulated by the liver. The enterohepatic circulation of bile acids and their excretion play decisive roles within a complex regulatory system. An average Western diet provides a cholesterol uptake of 0.3-0.5 g per day, and de novo synthesis contributes another 0.8 g per day. With a bile acid pool size of 2.5-5 g, the daily throughput of the enterohepatic circulation is 10-40 g of bile acids and about 2.5 g of cholesterol. By fecal excretion, 0.2-0.6 g of bile acids and 0.6-0.8 g of cholesterol are excreted per day. The liver can compensate for imbalances of the cholesterol level by various mechanisms. An increase of hepatic cholesterol can be achieved either by low-density lipoprotein (LDL) receptor induction and higher uptake from plasma, or by the stimulation of HMG-CoA reductase and increased de novo synthesis. A higher excretion is reached by increased conversion of cholesterol into bile acids or by elevated biliary cholesterol secretion. In humans, two different approaches have demonstrated that interruption of the enterohepatic circulation results in a significant decrease in serum cholesterol levels.

Bile acid sequestrants have been used to treat hypercholesterolemia for over 20 years. Anion exchange resins bind bile acids, thereby removing them from the enterohepatic circulation. This loss is compensated by resynthesis of bile acids from cholesterol. Much more dramatic were results obtained from the POSCH (Program on the Surgical Control of the Hyperlipidemics) study [39]: partial ileal bypass surgery in 421 patients led to a mean decrease of 23% total cholesterol and 37.7% LDL-cholesterol.

The disadvantages of sequestrants are related to the high dosages required (15-30 g per day), and side effects include constipation, indigestion, absorption and compliance problems. Thus, it was discovered that a more effective way of interrupting the enterohepatic circulation was the development of highly specific, non-absorbable inhibitors of the intestinal bile acid transport systems. Such bile acid reabsorption inhibitors have several advantages.

These pharmacological advantages include high specific mode of action, no systemic drug load and consequently no systemic toxicity, no mal-absorption and indigestion, low dosage and high compliance. It was then discovered that inhibition of the bile acid moiety should prevent transmembrane transport of the inhibitor itself across the ileal brush border membrane. As the ileal bile acid transport system is an oligomeric protein complex containing several transporter units, we developed the concept of linking bile acid molecules together to obtain dimers, trimers or tetramers. These molecules should inhibit ileal bile acid uptake by the simultaneous occupation of more than one transporter site, resulting in an efficient and specific inhibition of the ileal bile acid transport system without significant uptake of the inhibitor itself.

The present invention comprises novel processes for the preparation of bile acid reabsorption inhibitors through the synthesis of novel R and S chiral forms that are useful in the preparation of 2-bromomethyl-2-ethylhexanoic acid and related intermediates and derivatives for the treatment and regulation of metabolic disorders, diabetes, hyperglycemia and the like.

### SUMMARY OF THE INVENTION

The present invention comprises a novel process for the preparation of an R or S chiral compound of formula (I):

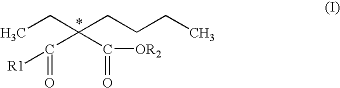

wherein R₁ is hydroxyl or an acid-activating functional group selected from the group consisting of chlorine and bromine radicals, hydroxyl-benzothiazole ester residues, mercapto-benzothiazole thioester residues, benzotriazole 3-oxide amide residues and mixed sulphonate- and phosphate-anhydride residues, and a group which activates the carboxyl and R₂ is alkyl optionally substituted by halogen or benzyl, its preparation, its application in the synthesis of chiral 2-bromomethyl-2-ethylhexanoic acid and novel intermediates useful in the synthesis of bile acid inhibitor compounds for the treatment of metabolic disorders.

### DETAILED DESCRIPTION OF THE INVENTION

The present invention relates to novel chiral compounds derived from hexanoic acid esters, methods for their preparation, chiral intermediates produced thereby and to their use in the synthesis of chiral 2-(bromomethyl)-2-ethylhexanoic acid, an intermediate in the syntheses of these bile acid resorption inhibitors.

A focal point of the subject matter of the present invention is an (R) or (S) chiral compound corresponding to the formula (I)

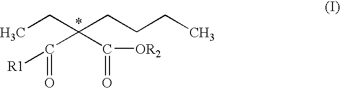

in which R₁ represents a hydroxyl radical or R′₁, R′₁ representing a group which activates the acid functional group, and R₂ represents an alkyl radical including from 1 to 8 carbon atoms, optionally substituted by one or more halogen atoms, or a benzyl radical.

The term “alkyl radical including from 1 to 8 carbon atoms” is understood to mean any type of linear or branched alkyl and preferably a methyl, ethyl or propyl or butyl radical which is linear or branched.

The term “halogen atom” is understood to mean the fluorine, chlorine, bromine or iodine atom.

The term “alkyl radical substituted by halogen” is understood to mean a methyl radical or, preferably, an ethyl radical substituted by one or more chlorine or fluorine atoms. The term “group which activates the acid functional group” is understood to mean any group known to a person skilled in the art, for example a chlorine or bromine atom or an ester residue, for example derived from 1-hydroxybenzotriazole, a thioester residue, for example derived from 2-mercaptobenzothiazole, an amide residue, for example derived from benzotriazole 3-oxide, or a mixed anhydride residue, for example derived from sulfonates or phosphates.

Such groups are known in particular in acylation processes.

A subject matter of the invention is in particular a compound of formula (I) as defined above, in which R₁ is chosen from the group consisting of a hydroxyl radical, a chlorine or bromine atom, a mixed anhydride residue, an activated thioester residue, an activated ester residue and an activated amide residue, and a compound of formula (I) as defined above, in which R₂ is chosen from the group consisting of an alkyl radical including from 1 to 4 carbon atoms and a benzyl radical.

Another subject matter of the invention is a process for the preparation of the compounds of formula (I) as defined above, which comprises the treatment of a compound of formula (II)

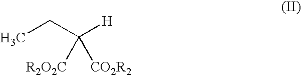

in which R₂ is defined as above,
with a reactant capable of attaching a chain of formula

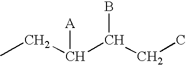

in which either A and B represent a hydrogen atom and C represents a bromine atom, or A and B form a second carbon-carbon bond and C represents a hydrogen atom, or A and C represent a hydrogen atom and B represents a ketone functional group,
in order to obtain a compound of formula (III):

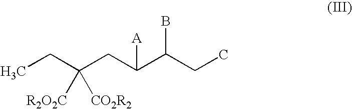

in which A, B, C and R₂ have the abovementioned meanings, the ketone functional group of which B may represent being, if appropriate, protected in order to obtain a compound of formula (III′)

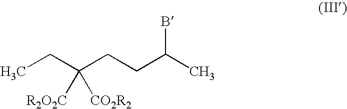

in which R₂ has the abovementioned meaning and B′ represents a protected ketone functional group, then the treatment of the compound of formula (III) or (III′) with an enzyme having a hydrolytic activity, in order to obtain a chiral compound of formula (IV):

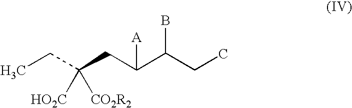

or a chiral compound of formula (IV₁):

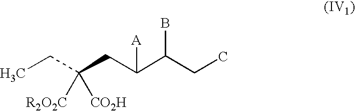

in which A, B, C and R₂ have the abovementioned meanings, or a corresponding chiral compound of formula (IV′) or (IV′₁)

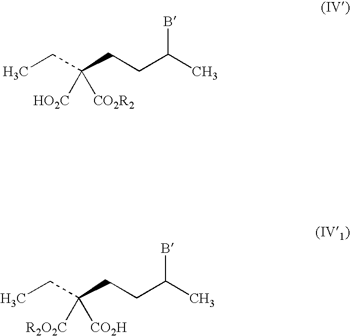

in which B′ and R₂ have the abovementioned meanings, which compound of formula (IV) or (IV₁) or (IV′) or (IV′₁) is treated with conditions capable of generating the corresponding chiral compound of formula (I_{A}):

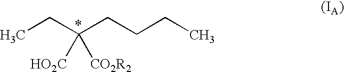

in which R₂ has the abovementioned meaning, corresponding to a compound of formula (I) in which R₁ is a hydroxyl radical, which compound, if appropriate, is treated with an agent which activates the acid functional group, in order to obtain a chiral compound of formula (I_{B})

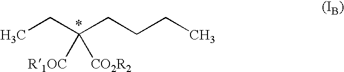

in which R₂ has the abovementioned meaning, corresponding to a compound of formula (I) in which R′₁ has the abovementioned meaning.

The reactant capable of attaching the chain of formula —CH₂—CHA-CHB—CH₂C is a halogenated derivative of said chain or a derivative unsaturated at the chain end. Examples appear below in the experimental part.

The protection of the ketone functional group which B may represent can be any protection known to a person skilled in the art, for example a ketal or a thioketal.

The enzyme having a hydrolytic activity bringing about asymmetry can be in particular an esterase, a protease or a lipase, for example a hog liver esterase, chymotrypsin or a hog pancreas lipase. Mention may in particular be made, among the preferred enzymes, of the semipurified hog liver enzyme known under the trade name chirazyme E₁.

The conditions capable of generating the chiral compound (I_{A}) depend, of course, on the nature of the compound employed. If it is a compound in which A and B form a second bond or a compound in which C represents a bromine atom, a reduction is carried out, for example with hydrogen in the presence of palladium in tetrahydrofuran. If it is a compound in which B represents a ketone functional group, a reduction of Wolff-Kishner type is carried out, for example. If it is a ketal, a reduction with sodium in liquid ammonia can be carried out. If it is a thioketal, a reduction with hydrogen in the presence of a metal catalyst, in particular nickel, can be carried out.

The agent which activates the acid functional group is an agent capable of forming either an acid chloride or bromide, or an ester, or a thioester, or an amide, or a mixed anhydride. Such agents are conventional and known to a person skilled in the art, for example in carrying out an acylation reaction.

A further subject matter of the invention is the application of the compounds of formula (I) as defined above in the preparation of the chiral compound of formula (A):

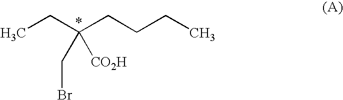

which comprises subjecting a compound of formula (I) to the action of a reducing agent in order to obtain a chiral compound of formula (V):

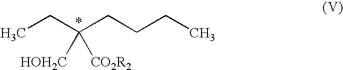

in which R₂ has the abovementioned meaning, which compound is saponified in order to obtain the chiral acid of formula (VI)

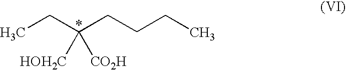

which compound is subjected to the action of a brominating agent in order to obtain the chiral compound of formula (A).

The reducing agent which is made to act on the compound (I) is, for example, an alkaline borohydride, such as sodium borohydride. It can also be an alkoxyborane or an acylborane.

The compound (I) used is preferably a compound in which R₁ represents R′₁.

The saponification of the compound of formula (V) can be carried out under conventional conditions known to a person skilled in the art.

The brominating agent is preferably hydrobromic acid.

The malonate derivative of formula (II) is known and described or can be prepared by described processes.

A final subject matter of the invention is the compounds of formula (III) and (III′) in which either A and B form a carbon-carbon bond and C represents a hydrogen atom, or A and C represent a hydrogen atom and B represents a ketone function, and B′ and R2 are as defined hereabove, the chiral compounds of formula (IV) and (IV₁), (IV′) and (IV′₁), and the chiral compounds of formula (V), (IV) and (A).

The compound of formula (A) is of use in particular in the synthesis of therapeutically active compounds.

It will be appreciated that every suitable combination of the compounds of the invention with one or more of the aforementioned compounds and optionally one or more other pharmacologically active substances is regarded as falling within the protection conferred by the present invention. The examples detailed below are provided to better describe and more specifically set forth the compounds, processes and methods of the present invention. It is to be recognized that they are for illustrative purposes only however, and should not be interpreted as limiting the spirit and scope of the invention as later recited by the claims that follow.

### Example 1

### (2R)-2-Ethyl-2-(methoxycarbonyl)hexanoic Acid

### Stage A

### Dimethyl 2-(4-bromobutyl)-2-ethylmalonate

10.6 g of dimethyl de-ethylmalonate, 57.4 g of 1,4-dibromobutane and 30 cm³ of tetrahydrofuran are mixed under an inert gas. The mixture is cooled to +3° C. and 8.5 g of potassium tert-butoxide in 55 cm³ of tetrahydrofuran are slowly introduced. After ¼ of an hour, the temperature is allowed to slowly rise and then the mixture is maintained at 25° C. with stirring for 3 h. It is poured into a mixture of 150 cm³ of water, 50 cm³ of methylene chloride and 5 cm³ of 2N hydrochloric acid, separation by settling is carried out and the organic phase is washed with water. The aqueous phases are reextracted with methylene chloride and the combined organic phases are dried and concentrated to dryness under reduced pressure. 62 g of an oil are obtained, which oil is distilled under a pressure of 1 mmHg. 16.2 g of the expected product are obtained.

NMR spectrum (CDCl₃): 250 MHz 3.71 ppm OCH₃ (6H, s), 3.42 ppm CH₂Br (2H, t), 1.9 ppm CH₂ (6H, m), 1.2 ppm CH₂ (2H, m), 0.8 ppm CH₃ (3H, t).

### Stage B

### (2R)-6-Bromo-2-ethyl-2-(methoxycarbonyl)hexanoic Acid

565 cm³ of water, 16.1 g of the product obtained in stage A and 28.5 cm³ of dimethyl sulfoxide are mixed, the mixture is heated to 30° C. and the pH is adjusted to 7 by addition of 1 N sodium hydroxide solution. 5.4 g of chirazyme E1 are added. The mixture is stirred for 30 h at approximately 30° C. while maintaining the pH at 6.75 and then the enzyme is filtered off. The enzyme is washed on the filter by gradual addition of 170 cm³ of water. The aqueous phase thus obtained is basified by addition of 0.92 g of sodium bicarbonate. The enzyme and the aqueous phases are subsequently washed with methylene chloride. The combination of the combined aqueous phases is acidified to pH 2.7 by addition of 30 cm³ of 2N hydrochloric acid. Extraction is carried out with isopropyl ether, the organic phase is washed with water, dried and concentrated to dryness under reduced pressure, and 12.9 g of the expected product are obtained.

NMR spectrum (CDCl₃) 250 MHz 3.79 ppm OCH₃ (3H, s), 3.40 ppm CH₂Br (2H, t), 2 ppm CH₂ (6H, m), 1.4 ppm CH₂ (2H, m), 0.8 ppm CH₃ (3H, t).

Ee=95% NMR CDCl₃ in the presence of (R)-methylbenzylamine

### Stage C

### (2R)-2-Ethyl-2-(methoxycarbonyl)hexanoic Acid

12.44 g of the product obtained in stage B, 125 cm³ of tetrahydrofuran, 2.5 g of 10% palladium-on-charcoal and 12.5 cm³ of triethylamine are mixed. The mixture is placed under a hydrogen atmosphere and is kept stirred at approximately 26° C. for 20 h. The catalyst is filtered off and is washed with tetrahydrofuran. The filtrate is concentrated to dryness under reduced pressure and the residue is taken up in 50 cm³ of isopropyl ether and 50 cm³ of water. The mixture is acidified by addition of 15 cm³ of 2N hydrochloric acid, separation by settling is carried out and the aqueous phase is reextracted with 50 cm³ of isopropyl ether. The combined organic phases are washed with water, dried and concentrated to dryness under reduced pressure. 8.91 g of the expected product are obtained.

NMR spectrum (CDCl₃) 250 MHz 3.79 ppm OCH₃ (3H, s), 2 ppm CH₂ (4H, m), 1.2 ppm CH₂ (4H, m), 0.8 ppm CH₃ (6H, td).

### Example 2

### Methyl (2S)-2-{[(diethoxyphosphoryl)oxy]carbonyl}-2-ethylhexanoate

8.5 g of the product obtained in Example 1, 42.5 cm³ of methylene chloride and 8.5 cm³ of diethyl chlorophosphate are mixed under an inert gas. 6.3 g of 2,6-lutidine in 8.5 cm³ of methylene chloride are slowly introduced at approximately 25° C. After stirring for 18 h at approximately 25° C., 35 cm³ of methylene chloride and 42.5 cm³ of water are added. The addition is carried out for 5 min, the layers are separated by settling and then the organic phase is washed with water. The organic phase is dried and concentrated to dryness under reduced pressure, and 16.96 g of the expected product are obtained, which product is stored in solution in 10 cm³ of methylene chloride.

NMR spectrum (CDCl₃) 250 MHz 4.3 ppm CH₂ (4H, q), 3.8 ppm OCH₃ (3H, s), 2 ppm CH₂ (4H, m), 1.4 ppm CH₂+CH₃ (10H, q), 0.8 ppm CH₃ (6H, t).

### Example 3

### (2R)-2-Ethyl-2-(methoxycarbonyl)hexanoic Acid

### Stage A

### Dimethyl 2-ethyl-2-(3-oxobutyl)malonate

10 cm³ of dimethyl 2-ethylmalonate, 20 cm³ of methanol and 2.5 cm³ of methyl vinyl ketone are mixed under an inert gas. 7.5 cm³ of methyl vinyl ketone and 1 cm³ of 10% sodium methoxide in methanol are introduced over 1 h. The mixture is subsequently kept stirred at 26-27° C., is cooled to approximately 15° C. and then 2 cm³ of 1 N hydrochloric acid and then 10 cm³ of water are added. The mixture is concentrated to half its volume, 90 cm³ of water are added and extraction is carried out with isopropyl ether. The organic phase is washed with water, dried and concentrated to dryness under reduced pressure, and 13.45 g of the expected product are obtained.

NMR spectrum (CDCl₃) 250 MHz 3.8 ppm OCH₃ (6H, s), 2.4 ppm CH₂ (2H, t), 2.2 ppm CH₂+CH₃ (5H, t+s), 2 ppm CH₂ (4H, q), 0.8 ppm CH₃ (3H, t).

### Stage B

### (2R)-2-Ethyl-2-(methoxycarbonyl)-5-oxohexanoic Acid

20 cm³ of water, 0.504 g of the product obtained in stage A and 2 cm³ of dimethyl sulfoxide are mixed. The mixture is kept stirred at approximately 33-35° C. and then 0.25 g of chirazyme E1 is slowly added while maintaining the pH at 7-7.3 by addition of 0.5N sodium hydroxide solution. After 2 h, 10 cm³ of methylene chloride are added, acidification is carried out to a pH of 2 by addition of 3 cm³ of 1 N hydrochloric acid, a further 10 cm³ of methylene chloride are added and separation is carried out by settling. The organic phase is washed with water, dried and concentrated to dryness under reduced pressure. 0.491 g of the expected product is obtained.

NMR spectrum (CDCl₃) 250 MHz 3.8 ppm OCH₃ (3H, s), 2.4 ppm CH₂ (2H, t), 2.2 ppm CH₂+CH₃ (5H, t+s), 1.9 ppm CH₂ (4H, q), 0.8 ppm CH₃ (3H, t).

Ee=96% NMR CDCl₃ in the presence of (R)-methylbenzylamine

### Stage C

### (2R)-2-Ethyl-2-(methoxycarbonyl)hexanoic Acid

0.25 g of the product obtained in stage B is mixed with 0.23 g of NH₂—NH—SO₂—C₆H₄—CH₃ and 2.5 ml of DMF. The mixture is left stirring for 2 hours and NaBH₃CN, in solution in DMF (2 ml), is added over 1 hour. After stirring for 24 h, the product is isolated by running into 10% aqueous NaHCO₃ solution and extraction is carried out in the presence of ethyl acetate to obtain, after concentrating, 0.2 g of the expected product.

NMR spectrum (CDCl₃) 250 MHz 3.79 ppm OCH₃ (3H, s), 2 ppm CH₂ (4H, m), 1.2 ppm CH₂ (4H, m), 0.8 ppm CH₃ (6H, td).

### Example 4

### (2R)-2-Ethyl-2-(methoxycarbonyl)hexanoic Acid

### Stage A

### Dimethyl 2-ethyl-2-[2-(2-methyl-1,3-dithiolan-2-yl)ethyl]malonate

4.65 g of the product obtained in stage A of Example 3 and 23 cm³ of toluene are mixed at 20-22° C. under an inert gas. 3.7 g of ethanedithiole and 4.25 g of boron trifluoride etherate are added over 10 min at approximately 23° C. After stirring for 17 h at ambient temperature, the reaction mixture is poured into a mixture of 50 cm³ of isopropyl ether and 50 cm³ of a water/ice mixture. Stirring is carried out for 5 min, separation by settling is carried out, the aqueous phase is reextracted with isopropyl ether and the combined organic phases are washed with water and with a 1% aqueous sodium bicarbonate solution. They are dried and concentrated to dryness under reduced pressure, and 6.64 g of the expected product are obtained.

NMR spectrum (CDCl₃) 250 MHz 3.8 ppm OCH₃ (6H, s), 3.3 ppm S—CH₂—CH₂—S (4H, m), 2.2 ppm CH₂—S (2H, m), 2 ppm CH₂ (2H, q), 1.8 ppm CH₂+CH₃ (5H, m+s), 0.9 ppm (3H, t).

### Stage B

### (2R)-2-Ethyl-2-(methoxycarbonyl)-4-(2-methyl-1,3-dithiolan-2-yl)butanoic Acid

6 cm³ of water, 0.124 g of the product obtained in stage A and 0.6 cm³ of dimethyl sulfoxide are mixed. 0.125 g of chirazyme E1 is slowly added while maintaining the temperature at 33-34° C. and the pH at 7.5-8.5 by addition of 0.2N sodium hydroxide solution. The reaction mixture is kept stirred for 26 h and then 6 cm³ of methylene chloride and 0.6 cm³ of 1 N hydrochloric acid are added. The mixture is separated by settling, the aqueous phase is reextracted with methylene chloride and the organic phases are combined, washed with water, dried and concentrated to dryness under reduced pressure. 0.107 g of the expected product is obtained.

NMR spectrum (CDCl₃) 250 MHz 3.8 ppm OCH₃ (3H, s), 3.3 ppm S—CH₂—CH₂—S (4H, m), 2.2 ppm CH₂—S (2H, m), 2 ppm CH₂ (2H, q), 1.8 ppm CH₂+CH₃ (5H, m+s), 0.9 ppm (3H, t).

Ee=95% NMR CDCl₃ in the presence of (R)-methylbenzylamine

### Stage C

### (2R)-2-Ethyl-2-(methoxycarbonyl)hexanoic Acid

0.107 g of the product obtained in stage B, 1 cm³ of tetrahydrofuran and 25 mg of nickel are mixed. The mixture is placed under a hydrogen atmosphere and is kept stirred at approximately 26° C. for 20 h. The catalyst is filtered off and is washed with tetrahydrofuran. The filtrate is concentrated to dryness under reduced pressure and the residue is taken up in 10 cm³ of isopropyl ether and 10 cm³ of water. Separation by settling is carried out and the aqueous phase is reextracted with 10 cm³ of isopropyl ether. The combined organic phases are washed with water, dried and concentrated to dryness under reduced pressure. 0.5 g of the expected product is obtained.

NMR spectrum (CDCl₃) 250 MHz 3.79 ppm OCH₃ (3H, s), 2 ppm CH₂ (4H, m), 1.2 ppm CH₂ (4H, m), 0.8 ppm CH₃ (6H, td).

### Example 5

### (2R)-2-Ethyl-2-(methoxycarbonyl)hexanoic Acid

### Stage A

### Dimethyl 2-ethyl-2-[2-(2-methyl-1,3-dioxolan-2-yl)ethyl]malonate

0.45 cm³ of ethylene glycol, 10 mg of para-toluenesulfonic acid and 0.88 cm³ of methyl orthoformate are mixed and then 0.92 g of the product obtained in stage A of Example 3 is added. The mixture is kept stirred for 24 h at ambient temperature and is then poured into 20 cm³ of a 1% aqueous sodium bicarbonate solution. Extraction is carried out with methylene chloride and the organic phase is washed with water, dried and concentrated to dryness under reduced pressure. 1.6 g of the expected product are obtained.

NMR spectrum (CDCl₃) 250 MHz 4 ppm O—CH₂—CH₂—O— (4H, s), 3.75 ppm OCH₃ (6H, s), 2 ppm CH₂ (4H, m), 1.5 ppm CH₂ (2H, m), 1.4 ppm CH₃ (3H, s), 0.9 ppm (3H, t).

### Stage B

### (2R)-2-Ethyl-2-(methoxycarbonyl)-4-(2-methyl-1,3-dioxolan-2-yl)butanoic Acid

20 cm³ of water, 0.55 g of the product obtained in stage A and 2 cm³ of dimethyl sulfoxide are mixed, the mixture is kept stirred at 30-32° C. and then 0.266 g of chirazyme E1 is slowly added while maintaining the temperature at approximately 33° C. and the pH at 7.2-7.5 by addition of 0.5N sodium hydroxide solution. After 24 h, the temperature is brought back to 20° C. and then 10 cm³ of methylene chloride are added. The mixture is acidified by addition of 2.3 cm³ of 1 N hydrochloric acid and separated by settling, and the organic phase is washed with water, dried and concentrated to dryness under reduced pressure. 0.452 g of the crude expected product is obtained.

NMR spectrum (CDCl₃) 250 MHz 4 ppm O—CH₂—CH₂—O— (4H, s), 3.8 ppm OCH₃ (6H, s), 2 ppm CH₂ (4H, m), 1.6 ppm CH₂ (2H, m), 1.4 ppm CH₃ (3H, s), 0.9 ppm CH₃ (3H, t).

Ee=99% NMR CDCl₃ in the presence of (R)-methylbenzylamine

### Stage C

### (2R)-2-Ethyl-2-(methoxycarbonyl)hexanoic Acid

0.4 g of the product obtained in stage B is mixed in 8 ml of liquid NH₃ at −70° C. with 0.22 mg of Na. The temperature is maintained at −70° C. for 3 h. An NH₄Cl solution (2 ml) is added over 1 h at −30° C. and extraction is carried out with ethyl acetate. The organic phase is washed with water, dried and concentrated to dryness under reduced pressure. 0.31 g of the expected product is obtained.

NMR spectrum (CDCl₃) 250 MHz 3.79 ppm OCH₃ (3H, s), 2 ppm CH₂ (4H, m), 1.2 ppm CH₂ (4H, m), 0.8 ppm CH₃ (6H, td).

### Example 6

### (2R)-2-Ethyl-2-(methoxycarbonyl)hexanoic Acid

### Stage A

### Dimethyl (2E)-2-[but-2-enyl]-2-ethylmalonate

10 cm³ of dimethyl 2-ethylmalonate, 20 cm³ of DMF and 1.27 g of NaH are mixed at 0° C. under an inert gas. 5.8 g of 4-chloro-2-butene are introduced over 1 h while maintaining the temperature at 0° C. After reacting for 2 h at 0° C., 10 ml of 1 N HCl are introduced, the mixture is then concentrated, 10 ml of water are added and extraction is carried out with isopropyl ether. After dry extract, 8.2 g (E/Z 85/15) of an oil are recovered.

NMR spectrum (CDCl₃) 250 MHz 4.6 ppm vinyl H(H, td), 4.2 ppm vinyl H (H, q), 3.75 ppm OCH₃ (6H, s), 2.5 ppm CH₂ (2H, dd), 2.4 ppm CH₃ (3H, d), 1.4 ppm CH₂ (2H, q), 0.9 ppm (3H, t).

### Stage B

### (2R,4E)-2-Ethyl-2-(methoxycarbonyl)hex-4-enoic Acid

8 g of the product obtained in stage A are mixed. 8 g of chirazyme E1 are slowly added while maintaining the temperature at approximately 33° C. and the pH at 6.88 by addition of 1 N sodium hydroxide solution. After 24 h, extraction is carried out with methylene chloride and the organic phase is washed with water, dried and concentrated to dryness under reduced pressure. 6.8 g of the expected product are obtained.

NMR spectrum (CDCl₃) 250 MHz 4.6 ppm vinyl H(H, td), 4.2 ppm vinyl H (H, q), 3.70 ppm OCH₃ (3H, s), 2.6 ppm CH₂ (2H, dd), 2.5 ppm CH₃ (3H, d), 1.5 ppm CH₂ (2H, q), 0.9 ppm (3H, t).

Ee=26% NMR CDCl₃ in the presence of (R)-methylbenzylamine

### Stage C

### (2R)-2-Ethyl-2-(methoxycarbonyl)hexanoic Acid

6 g of the product obtained in stage B, 60 cm³ of tetrahydrofuran, 1.25 g of 10% palladium-on-charcoal and 6.25 cm³ of triethylamine are mixed. The mixture is placed under a hydrogen atmosphere and is kept stirred at approximately 26° C. for 20 h. The catalyst is filtered off and is washed with tetrahydrofuran. The filtrate is concentrated to dryness under reduced pressure and the residue is taken up in 25 cm³ of isopropyl ether and 25 cm³ of water. The mixture is acidified by addition of 7 cm³ of 2N hydrochloric acid, separation by settling is carried out and the aqueous phase is reextracted with 25 cm³ of isopropyl ether. The combined organic phases are washed with water, dried and concentrated to dryness under reduced pressure. 5.7 g of the expected product are obtained.

NMR spectrum (CDCl₃) 250 MHz 3.79 ppm OCH₃ (3H, s), 2 ppm CH₂ (4H, m), 1.2 ppm CH₂ (4H, m), 0.8 ppm CH₃ (6H, td).

### Example 7

### (2S)-2-(Bromomethyl)-2-ethylhexanoic Acid

### Stage A

### Methyl (2R)-2-ethyl-2-(hydroxymethyl)hexanoate

27.4 g of the mixed anhydride solution obtained in Example 2 are concentrated to 16 g and then 53 cm³ of dimethylformamide are added thereto with stirring and under an inert gas. The mixture is cooled to approximately +2° C. and then 1.75 g of sodium borohydride are slowly added. 1 h 45 min after the end of the introduction, a further 0.17 g of sodium borohydride is added and then, 1 h later, a further 0.17 g of sodium borohydride is again added. 73 cm³ of isopropyl ether are subsequently added and then 35 cm³ of a 5% aqueous tartaric acid solution are added at 6-10° C. over 15 min. After stirring for 5 min, the mixture is separated by settling, the aqueous phase is reextracted with isopropyl ether and then the combined organic phases are washed with a saturated aqueous sodium bicarbonate solution and then with water. They are dried and concentrated to dryness under reduced pressure, and 7.3 g of the crude expected product are obtained, which product is chromatographed on silica, elution being carried out with a heptane/ethyl acetate 7/3 mixture. 7.2 g of the purified product are obtained.

NMR spectrum (CDCl₃) 250 MHz 3.79 ppm OCH₃ (3H, s), 3.69 ppm CH₂OH (2H, s), 2 ppm CH₂ (4H, m), 1.2 ppm CH₂ (4H, m), 0.8 ppm CH₃ (6H, td).

### Stage B

### (2R)-2-Ethyl-2-(hydroxymethyl)hexanoic Acid

7 g of the product obtained in stage A are dissolved in 70 ml of methanol, and 37 ml of 1 N sodium hydroxide solution are added at 0° C. The mixture is maintained at 0° C. for 1 h; the medium is concentrated, the residue is taken up in 37 ml of 1 N HCl and the expected product is extracted with 2×50 ml of ethyl acetate. 5.6 g of the expected product are obtained.

NMR spectrum (CDCl₃) 250 MHz 3.69 ppm CH₂OH (2H, s), 1.6 ppm CH₂ (4H, m), 1.2 ppm CH₂ (4H, m), 0.8 ppm CH₃ (6H, td).

### Stage C

### (2S)-2-(Bromomethyl)-2-ethylhexanoic Acid

5.6 g of the product obtained as described in stage B and 34.8 cm³ of 62% hydrobromic acid are mixed, the mixture is then brought to 92° C.±2° C. with stirring for 7 hours and then it is left standing for 16 hours at 20° C. The mixture is cooled to 0° C. and 60 cm³ of water and then 9.8 cm³ of 32% sodium hydroxide solution are added. The mixture is kept stirred and 25 cm³ of toluene are introduced, then 50 cm³ of water and 50 cm³ of toluene are again introduced and the mixture is stirred for 1 hour at 20° C. The mixture is separated by settling, the aqueous phase is reextracted with toluene and the organic phases are combined, dried and concentrated to dryness under reduced pressure. 6.1 g of the crude expected product are obtained, which product is purified by distillation under 2 mmHg. 3.17 g of the expected product are obtained (Bp₂=118-124° C.).

NMR spectrum (CDCl₃) 250 MHz 3.52 ppm CH₂Br (2H, s), 1.6 ppm CH₂ (4H, m), 1.2 ppm CH₂ (4H, m), 0.8 ppm CH₃ (6H, td).

α^{D}₍₂₀₎ (1% CHCl₃)=+4°

What we claim is:

1. A process for the preparation of an R or S chiral compound of formula (I) 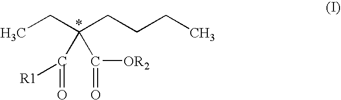
in which R1 represents a hydroxyl radical or R′₁, wherein R′₁ is an acid-activating functional group selected from the group consisting of chlorine and bromine radicals, hydroxyl-benzothiazole ester residues, mercapto-benzothiazole thioester residues, benzotriazole 3-oxide amide residues and mixed sulphonate- and phosphate-anhydride residues, and
R₂ is a C1-C8-alkyl, optionally substituted by one or more halogen atoms, or a benzyl radical comprising:
a) treating a compound of formula (II) 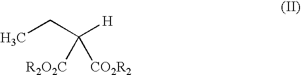
with a reactant capable of attaching a chain represented by the formula 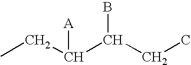
where said reactant is halogenated at the chain end or unsaturated at the chain end; and
wherein either A and B is hydrogen and C is bromine, or
A and B form a second carbon-carbon bond and C is hydrogen, or
A and C are both hydrogen and B is a ketone functional group producing a compound of formula (III) 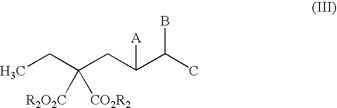
wherein A, B, C and R₂ are as defined above and the ketone functional group of B is optionally protected in order to obtain a compound of formula (III′) 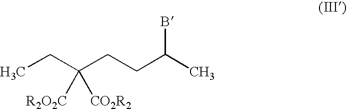
wherein R₂ is as defined above and B′ is a protected ketone functional group selected from the group consisting of ketal and thioketal,
b) treating the compound of formula (III) or (III′) with an hydrolytic enzyme selected from the group consisting of an esterase, a protease, a lipase, a hog liver esterase, chymotrypsin, a hog pancreas lipase, chirazyme E₁, and mixtures thereof, to produce a chiral compound of formula (IV): 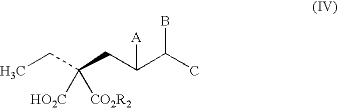
or a chiral compound of formula (IV₁): 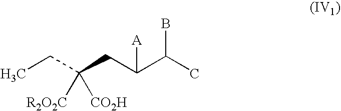
or the compound of formula (IV′) or (IV′₁) 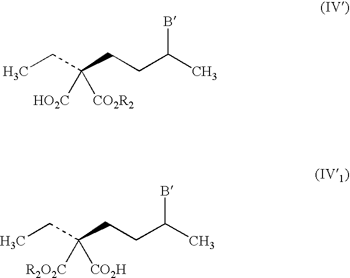
wherein A, B, C, R₂ and B′ are as defined above;
c) treating the compounds of formula (IV) or (IV₁) or (IV′) or (IV′₁) under conditions capable of generating the corresponding chiral compound of formula (I_{A}): 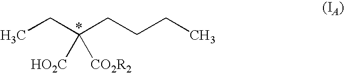
wherein R₂ is as defined above and R₁ is hydroxyl and,
d) optionally treating a compound of formula (I_{A}) with an agent which activates the acid functional group, in order to obtain a chiral compound of formula (I_{B}) 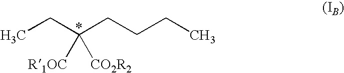
wherein R′₁ and R₂ are defined above.

2. A process for the preparation of the chiral compound of formula (A): 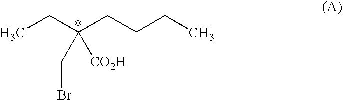
comprising:
a) reacting a compound of formula (I) as recited in claim 1 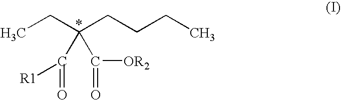
wherein R₁ is hydroxyl or R′₁ wherein R′₁ is an acid activating functional group selected from the group consisting of chlorine and bromine radicals, hydroxybenzothiazole-derived ester residues, mercaptobenzothiazole-derived thioester residues, benzotriazole 3-oxide derived amide residues and mixed sulphonate- and phosphate-derived anhydride residues and R₂ is a C1-C8-alkyl, optionally substituted by one or more halogen atoms, or a benzyl group;
with a reducing agent selected from the group consisting of an alkaline borohydride, an alkoxyborane or an acylborane in order to obtain a chiral compound of formula (V): 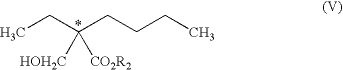
wherein R₂ is C1-C8-alkyl, optionally substituted by one or more halogen atoms, or a benzyl group;
b) saponifying the compound of formula (V) in order to obtain the chiral acid of formula (VI) 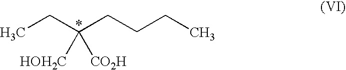
c) subjecting the compound of formula (VI) to a brominating agent in order to obtain the chiral compound of formula (A).

3. The process of claim 2 wherein R₁ of formula (I) is R′₁.

4. A compound selected from the group consisting of: 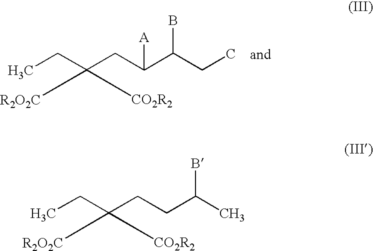
wherein:
i) A and B form a carbon-carbon bond and C is hydrogen, or
ii) A and C are both hydrogen and B is a ketone functional group, B′ is a protected ketone functional group selected from the group consisting of ketal and thioketal and R₂ is C₁-C₈-alkyl, optionally substituted by one or more halogens, or a benzyl radical.

5. A chiral compound selected from the group consisting of: 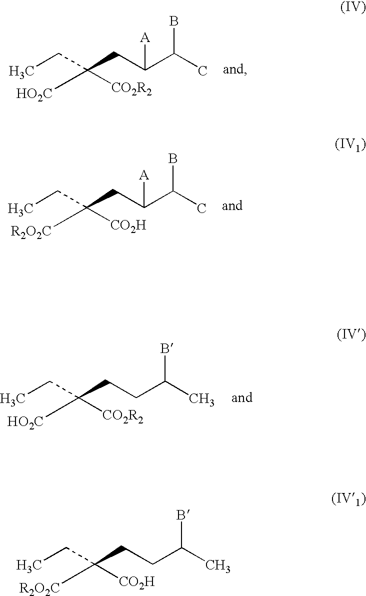
wherein:
i) A and B are both hydrogen and C is a bromine atom, or,
ii) A and B form a second carbon-carbon bond and C is a hydrogen, or
iii) A and C each is hydrogen atom and B is a ketone functional group, and
iv) R₂ is C1-C8-alkyl, optionally substituted by one or more halogen atoms, or a benzyl group, and
v) B′ is a protected ketone functional group selected from the group consisting of a ketal and a thioketal.
